(19) United States
(12) Patent Application Publication
(10) Pub. No.: US 2008/0249506 A1
(43) Pub. Date: Oct. 9, 2008

(54) Methods of treating diverticula

(76) Inventors: David Neustaedter, Lynn, MA (US); Allen Poma, Somerville, MA (US)

Correspondence Address: MCCRACKEN & FRANK LLP, 311 S. WACKER DRIVE, SUITE 2500, CHICAGO, IL, 60606, US

(21) Appl. No.: 12/080,732

(22) Filed: Apr. 4, 2008

Related U.S. Application Data
(60) Provisional application No. 60/922,091, filed on Apr. 6, 2007.

(51) Int. Cl. A61M 31/00

(52) U.S. Cl. 604/514

(57) ABSTRACT

A method for treating a diverticulum includes the step of examining a diverticulum by inserting an endoscope into a body cavity. The method further includes the step of applying a material to the diverticulum with the endoscope.

### CROSS REFERENCE TO RELATED APPLICATIONS

This application claims the benefit of U.S. Provisional Application No. 60/922,091 filed Apr. 6, 2007 entitled “Apparatuses and Methods of Treating Diverticula”.

### REFERENCE REGARDING FEDERALLY SPONSORED RESEARCH OR DEVELOPMENT

Not applicable

### SEQUENTIAL LISTING

Not applicable

### BACKGROUND OF THE INVENTION

1. Field of the Application

The present application relates generally to apparatuses and methods for treating diverticula, and more particularly to apparatuses and methods of filling, reducing, or blocking diverticula to prevent infection therein.

2. Description of the Background

In some areas of the world, such as Africa, diverticula, which are pouches or sacs branching out from a hollow organ or structure such as the intestine or colon, are rarely seen. Many researchers believe this difference is due to differences in diet. The colon functions to process otherwise indigestible dietary fiber. In areas of the world with a higher prevalence of diverticulitis, typically, less fiber is consumed as compared to areas, as opposed with lower prevalence of diverticulitis. When a substantial amount of fiber is processed by the colon, such as in a vegetarian diet, the contents of the colon keep the walls of the bowel apart. In contrast, if little fiber is digested, and thus processed, the contents of the colon tend to be smaller and harder, thus tending to become trapped in one or more diverticula, potentially causing infection and inflammation.

Diverticula may occur anywhere from the esophagus to the colon, but are most common in the colon. The prevalence of diverticula increases with age, with diverticula being uncommon before forty years of age, but likely present in a majority of Americans by the time of death. Such diverticula often become infected and/or inflamed, resulting in the condition of diverticulitis.

Physicians and scientists have developed various methods for treating diverticula both before and after diverticulitis has occurred or may occur. Many physicians, upon discovering diverticula that are not yet infected or inflamed, recommend a high fiber diet, often supplemented with bran or psyllium, to prevent further diverticula from forming. In order to treat active diverticulitis, physicians will commonly prescribe antibiotics. While these drugs are often efficacious in treating the episode, infected diverticula can be closed off and difficult to penetrate rapidly with a systemic therapy, requiring emergency surgical intervention. In the past, some attempts were made to surgically remove impacted material from diverticula, but such a surgery may have to be frequently repeated.

In many cases, a recommendation is made to remove the diverticula in order to prevent future episodes and the risk of complications such as sepsis. In order to remove diverticula, physicians remove a portion of the organ in which the diverticula are located, most frequently one or more segments of the colon. Such a procedure is a major surgery, and thus has often been limited to cases in which diverticulitis has recurred. While this therapy, colectomy, substantially reduces the frequency of recurrence, this approach results in significant healthcare costs, substantial pain, and morbidity for patients, and a significant mortality rate.

### SUMMARY OF THE INVENTION

According to one aspect of the present invention, a method for treating a diverticulum includes the step of examining a diverticulum by inserting an endoscope into a body cavity. The method further includes the step of applying a material to the diverticulum with the endoscope.

According to another aspect of the present invention, a method of treating a diverticulum in a body cavity or passageway includes the steps of inserting an endoscope into the cavity and pulling walls forming the diverticulum toward one another utilizing the endoscope. The method further includes the step of applying adhesive to the walls forming the diverticulum to secure the walls to one another.

According to a further aspect of the present invention, a method of treating diverticula in a body cavity or passageway includes the step of inserting a cylindrical structure into the body cavity such that solid portions of the cylindrical structure align with openings of two or more diverticula. The solid portions block the openings of the diverticula to prevent material from entering the diverticula.

Other aspects and advantages of the present application will become apparent upon consideration of the following detailed description.

### BRIEF DESCRIPTION OF THE DRAWINGS

FIG. 1 depicts a gastrointestinal tract of a biological organism having diverticula;

FIG. 2 is a cross-sectional view of a hollow organ of a biological organism in which diverticula have formed, wherein a first embodiment of an apparatus and method of treating diverticula is employed;

FIG. 3 is a cross-sectional sagittal view similar to that of FIG. 2, wherein a second embodiment of an apparatus and method of treating diverticula is employed;

FIG. 4 is a cross-sectional view similar to that of FIG. 2, wherein a third embodiment of an apparatus and method of treating diverticula is employed;

FIG. 5 is a cross-sectional view similar to that of FIG. 2, wherein a fourth embodiment of an apparatus and method of treating diverticula is employed;

FIGS. 6-9 are cross-sectional views depicting a fifth embodiment of an apparatus and method of treating diverticula; and

FIGS. 10 and 11 are cross-sectional views depicting a sixth embodiment of an apparatus and method of treating diverticula.

Throughout the figures, like or corresponding reference numerals have been used for like or corresponding parts.

### DETAILED DESCRIPTION

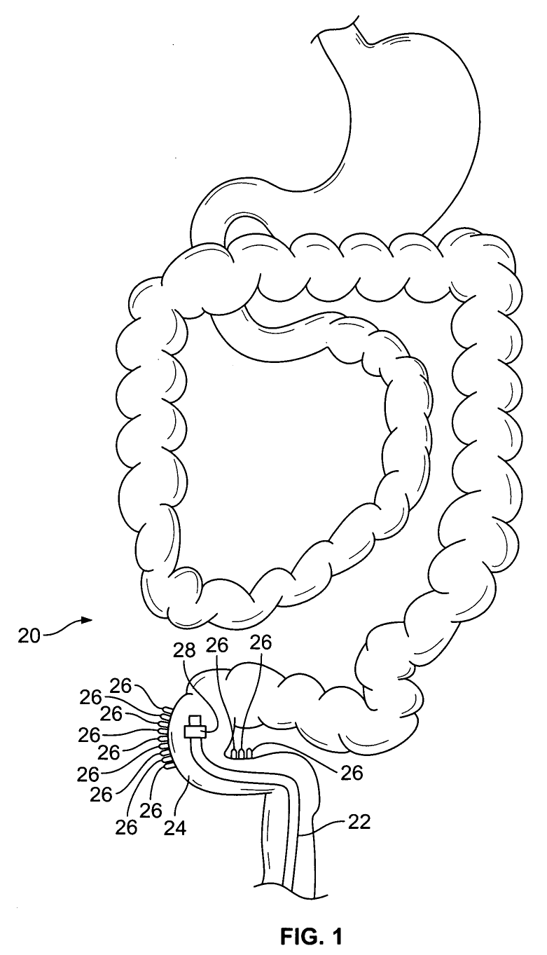

FIG. 1 depicts a gastrointestinal tract 20 of a biological organism, in particular, a human being. Although the embodiments herein will be described with respect to use in a human being, such embodiments may be utilized in any living organism. In the embodiments of the present application, an elongate tube 22 is inserted into a body cavity or passageway 24, such as a colon, intestine, esophagus, etc. within the biological organism to treat diverticula 26. The tube 22 may be an endoscope or the like that allows a physician to view, examine, and/or treat body cavities 24. In addition, the tube 22 preferably includes one or more attachment instruments 28 that may include a light, a plurality of optical fibers, a suction tube, a surgical tool, etc. for viewing, examining, and/or manipulating the diverticula 26.

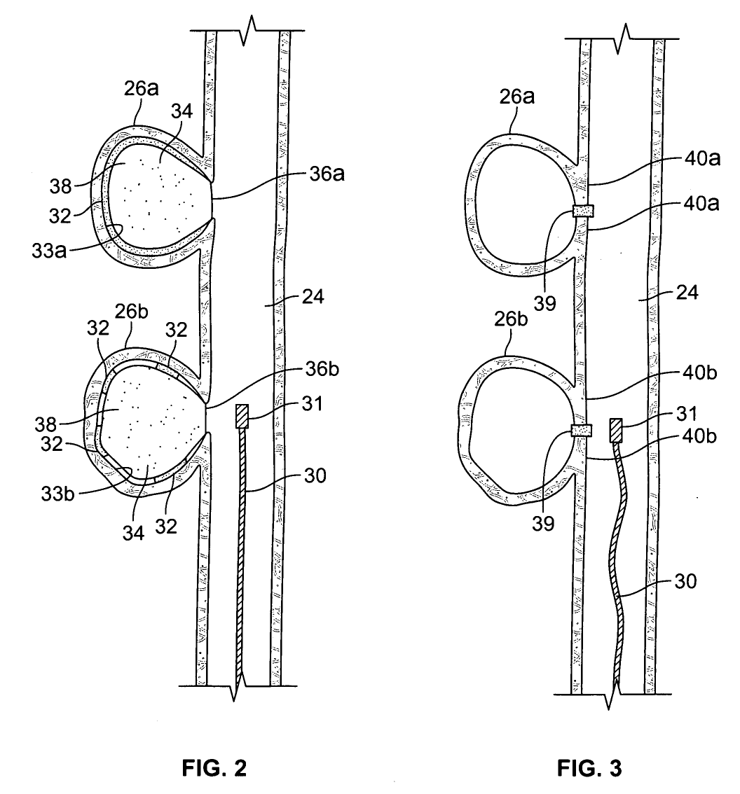

FIG. 2 depicts a first embodiment of an apparatus and method of treating diverticula 26a, 26b in a body cavity 24. The body cavity 24 may be any cavity in which diverticula 26a, 26b form. An endoscope 30 with attachments 31 for cleaning, dispensing adhesive, and dispensing a filler material is inserted into the body cavity 24. An adhesive 32 is inserted into the diverticula 26a, 26b and thereafter, a filler material 34 is inserted into the diverticula 26a, 26b. Optionally, the adhesive may be inserted into the diverticula 26a, 26b at the same time as the filler material 34. Still optionally, the adhesive 32 may be mixed within the filler material 34 or the filler material 34 may have adhesive properties such that the filler material 34 adheres to the diverticula walls and a separate adhesive is not necessary. Also optionally, the endoscope 30 may include a cleaning attachment for cleaning the diverticula 26a, 26b before inserting the adhesive 32 and filler material 34 into the diverticula 26a, 26b. The adhesive 32 can be any adhesive component that will allow the filler material to adhere to the inner surfaces 33a, 33b of the diverticula 26a, 26b and/or to seal openings 36a, 36b of the diverticula 26a, 26b.

Suitable adhesive agents could include, but are not limited to, hyaluronic acid, fibrin glue, fibrin clot, blood clot, collagen gel, alginate gel, gelatin-resorcin-formalin adhesive, mussel-based adhesive, dihydroxyphenylalanine (DOPA) based adhesive, chitosan, transglutaminase, poly(amino acid)-based adhesive, cellulose-based adhesive, polysaccharide-based adhesive, synthetic acrylate-based adhesives, platelet rich plasma (PRP), platelet poor plasma (PPP), clot of PRP, clot of PPP, Matrigel, Monostearoyl Glycerol co-Succinate (MGSA), Monostearoyl Glycerol co-Succinate/polyethylene glycol (MGSA/PEG) copolymers, laminin, elastin, proteoglycans, divinyl sulfone (DVS), polyethylene glycol divinyl sulfone (VS-PEG-VS), hydroxyethyl methacrylate divinyl sulfone (HEMA-DIS-HEMA), formaldehyde, glutaraldehyde, aldehydes, isocyanates, alkyl and aryl halides, imidoesters, N-substituted maleimides, acylating compounds, carbodiimide, hydroxychloride, N-hydroxysuccinimide, and combinations thereof. The adhesive 32 may be applied to an entire inner surface 33 of the diverticula 26a, 26b as seen in diverticulum 26a or the adhesive may be applied to portions of the inner surface 33 of the diverticula 26a, 26b as seen in diverticulum 26b.

The filler material 34 may be particulate, fibrous, organic, inorganic or a mixture of organic and inorganic. Suitable fillers include synthetic polymers derived from vinyl, acrylate, methacrylate, urethane, ester and oxide monomers, naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonia alginate, sodium, potassium or calcium alginate, propylene glycol alginate; gelatin; collagen; and cellulosics such as ethyl cellulose (EC), methylethyl cellulose (MEC), Carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), cellulose acetate phthalate (CAP), cellulose acetate trimellitate (CAT), hydroxypropylmethyl cellulose (HPMC), hydroxypropylmethyl cellulose phthalate (HPMCP), hydroxypropylmethyl cellulose acetate succinate (HPMCAS), hydroxypropylmethyl cellulose acetate trimellitate (HPMCAT), and ethylhydroxy ethylcellulose (EHEC). Other materials useful as the filler include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

Optionally, an antibiotic 38 can be mixed in the adhesive 32 and/or the filler material 34 to treat and/or prevent infection in the diverticula 26a, 26b.

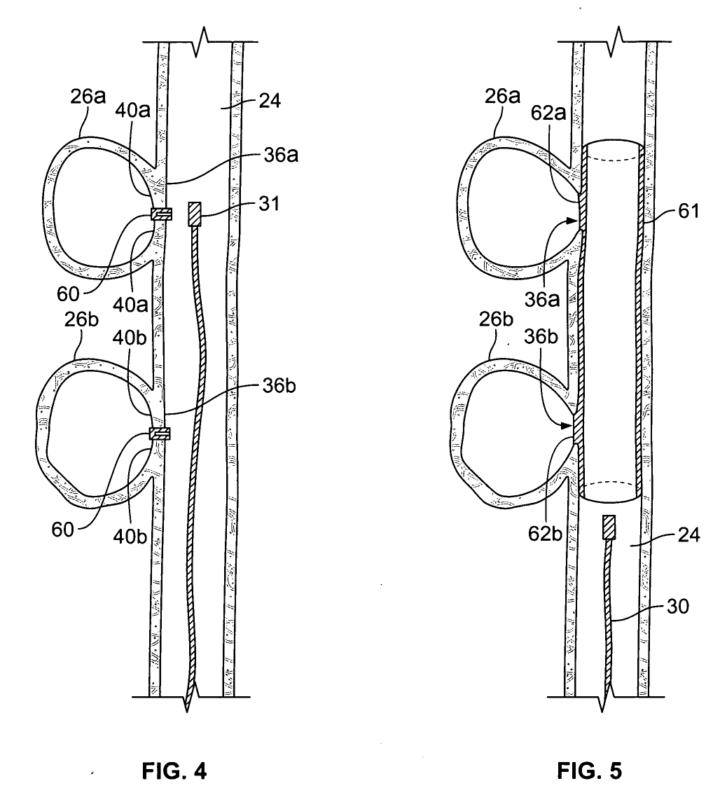

A second embodiment of an apparatus and method of treating diverticula 26a, 26b in a body cavity 24 is shown in FIG. 3. The body cavity 24 may be any cavity in which diverticula 26a, 26b develop. An endoscope 30 with attachments 31 for cleaning and for dispensing adhesive is inserted into the body cavity 24. Prior to treatment of the diverticula 26a, 26b, the diverticula may 26a, 26b be cleaned. Thereafter, the adhesive 39 is deposited in openings 36a, 36b of the diverticula 26a, 26b, wherein the endoscope 30 or another tool may aid in pulling neck portions 40a, 40b of the diverticula 26a, 26b inwardly such that the adhesive 32 seals the neck portions 40a, 40b to close the openings 36a, 36b. As with the previous embodiment, the adhesive 32 may include an antibiotic mixed therein to treat and/or prevent infection in the diverticula 26a, 26b. FIG. 4 depicts an embodiment of an apparatus and method for treating diverticula 26a, 26b in a body cavity 24, wherein the apparatus and method are similar to those of the embodiment of FIG. 3. In a third embodiment of FIG. 4, the endoscope 30 includes an attachment 31 for pulling neck portions 40a, 40b of the diverticula 26a, 26b together and attaching a suture or staple 60 through the neck portions 40a, 40b to seal the openings 36a, 36b and prevent material from entering the diverticula 26a, 26b.

A fourth embodiment of an apparatus and method of treating diverticula 26a, 26b in a body cavity 24, as shown in FIG. 5, includes a stent, tube, or other generally cylindrical structure 61, whether solid or broken by an opening or openings, is inserted into the body cavity 24 in which the diverticula 26a, 26b have developed. The cavity 24 may be any cavity 24 in which diverticula 26a, 26b may develop. Preferably, an endoscope 30 with a cleaning attachment is used to clean the cavity 24 and/or diverticula 26a, 26b prior to treatment of the diverticula 26a, 26b. An endoscope 30 or other device is thereafter utilized to insert the cylindrical structure 61 into the cavity 24. In one example, once the stent 61 is inserted into the cavity 24, the stent 61 expands to lock itself in place within the cavity 24. The cylindrical structure 61 includes solid areas 62a, 62b that align with the openings 36a, 36b of the diverticula 26a, 26b. Although the cylindrical structure 61 may be used to block off a single diverticulum 26, the cylindrical structure 61 is preferably used to block off multiple diverticula 26a, 26b.

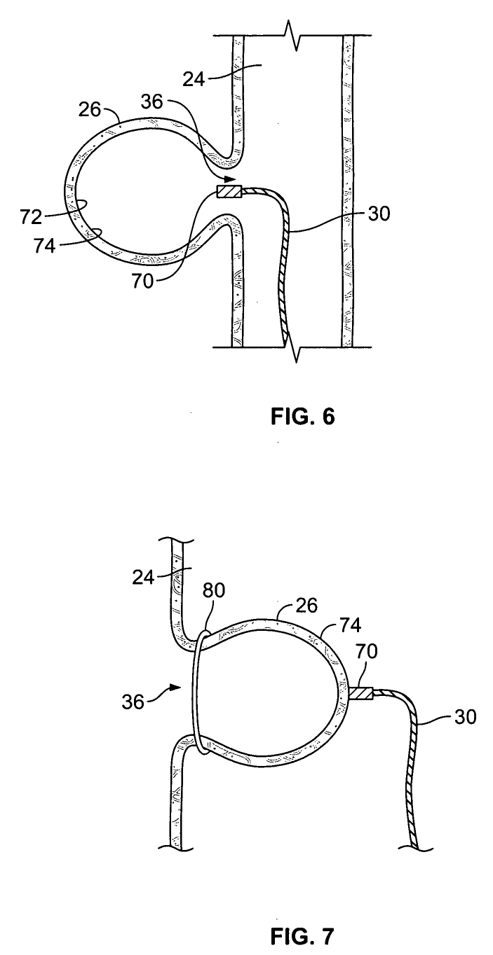

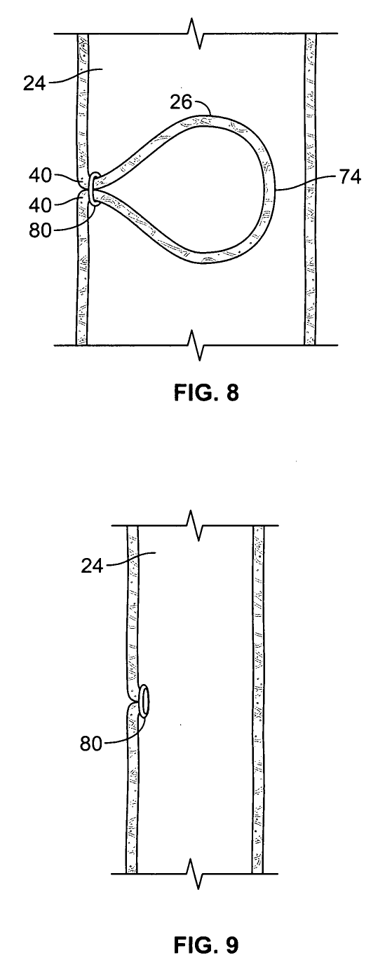

FIGS. 6-9 depict a fifth embodiment of an apparatus and method of treating a diverticula 26 in a body cavity 24. Although FIGS. 6-9 depict a single diverticulum 26, such an apparatus and method may be utilized on any number of diverticula 26. As seen in FIG. 6, an endoscope 30 with an attachment 31 containing a vacuum 70 for applying suction is inserted into each of the diverticula 26 to be treated. The vacuum 70 is turned on at the opening 36 of each diverticula 26 or near a distal end 72 of the diverticula 26. The suction from the vacuum 70 causes an inner wall 74 of the diverticula 26 to adhere to the vacuum 70 such that a physician may thereafter begin withdrawing the vacuum 70 from the cavity 24. Alternatively, the distal end 72 of the diverticula can be mechanically fixed by a clasping device that either by pinching together material and/or by penetrating the material of the diverticula achieves a strong attachment to the wall of the diverticula. Upon withdrawal of the vacuum 70, the diverticula 26 becomes inverted, as seen in FIG. 7. As the diverticula 26 becomes inverted, another endoscope or other device is used to fasten an attachment 80 around the opening 36 of the inverted diverticula 26, as further seen in FIG. 7. The attachment 80 may be a suture, a clip, a rubber band, a staple or the like. As the attachment 80 is fastened around the opening 36 of the diverticula 36, neck portions 40 of the diverticula 26 are moved toward one another to block the openings 36 of the diverticula 26, as seen in FIG. 8. Thereafter, as seen in FIG. 9, the inverted diverticula 26 are clipped, cut, or sutured to completely remove the diverticula 26. After removing the diverticula 26, the clipped, cut, or sutured areas are left to heal and the attachment 80 may be removed after healing for a period of time. Optionally, the attachment 80 may be an absorbable suture, clip, band, etc. that dissolves over time, thereby eliminating the need to remove the attachment 80. Also optionally, instead of clipping, cutting, or suturing the inverted diverticula 26, a substance may be used to coat the inverted diverticula 26, wherein the substance gradually reduces the size of and eventually destroys the diverticula 26.

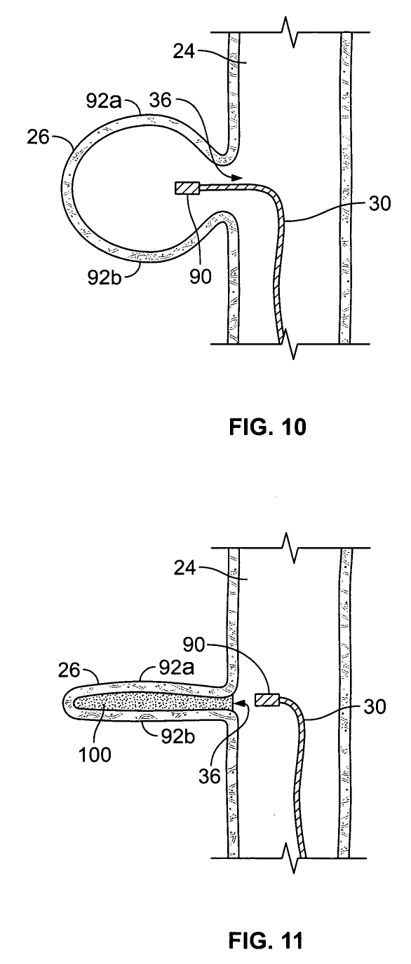

A sixth embodiment of an apparatus and method for treating diverticula 26 in a body cavity 24 is depicted in FIGS. 10 and 11. As seen in FIG. 10, an endoscope 30 with an attachment 90 for pulling walls 92a, 92b of the diverticula 26 together is inserted into the cavity 24 and the diverticula 26. The attachment 90 may be a vacuum or any mechanical attachment that would allow the walls 92a, 92b to be gripped and pulled together. Once the walls 92a, 92b are pulled together, the same or a different endoscope 30 with an attachment for depositing adhesive 100 is used to insert adhesive between the walls 92a, 92b to block off openings 36 of the diverticula 26.

Although a number of diverticula are depicted in each of the embodiments herein, the apparatuses and methods as disclosed herein may be used on one or more diverticula. In addition, any number of the above-described apparatuses and methods may be combined in any fashion to treat diverticula.

Numerous modifications to the present application will be apparent to those skilled in the art in view of the foregoing description. Accordingly, this description is to be construed as illustrative only and is presented for the purpose of enabling those skilled in the art to make and use the embodiments of the present application and to teach the best mode of carrying out same.

We claim:

1. A method for treating a diverticulum, the method comprising the steps of:
examining a diverticulum by inserting an endoscope into a body cavity; and
applying a material into the diverticulum with the endoscope.

2. The method of claim 1, further including the step of pulling a neck portion of the diverticulum together to close an opening of the diverticulum.

3. The method of claim 2, further including the step of attaching a staple or a suture to the neck portion of the diverticulum that has been pulled together to close the opening of the diverticulum.

4. The method of claim 1, further including the steps of cleaning the diverticulum with the endoscope, applying an antibiotic to the diverticulum, and sealing walls of the diverticulum together with an adhesive.

5. The method of claim 4, wherein the adhesive includes one or more of hyaluronic acid, fibrin glue, fibrin clot, blood clot, collagen gel, alginate gel, gelatin-resorcin-formalin adhesive, mussel-based adhesive, dihydroxyphenylalanine (DOPA) based adhesive, chitosan, transglutaminase, poly(amino acid)-based adhesive, cellulose-based adhesive, polysaccharide-based adhesive, synthetic acrylate-based adhesives, platelet rich plasma (PRP), platelet poor plasma (PPP), clot of PRP, clot of PPP, Matrigel, Monostearoyl Glycerol co-Succinate (MGSA), Monostearoyl Glycerol co-Succinate/polyethylene glycol (MGSA/PEG) copolymers, laminin, elastin, proteoglycans, divinyl sulfone (DVS), polyethylene glycol divinyl sulfone (VS-PEG-VS), hydroxyethyl methacrylate divinyl sulfone (HEMA-DIS-HEMA), formaldehyde, glutaraldehyde, aldehydes, isocyanates, alkyl and aryl halides, imidoesters, N-substituted maleimides, acylating compounds, carbodiimide, hydroxychloride, and N-hydroxysuccinimide.

6. The method of claim 1, further including the step of inserting a cylindrical structure into the body cavity to block off the diverticulum.

7. The method of claim 1, further including the steps of applying a vacuum suction at an opening of the diverticulum to invert the diverticulum and fastening an attachment around a neck of the diverticulum.

8. The method of claim 7, further including the step of removing the inverted diverticulum.

9. The method of claim 7, further including the step of applying a substance to the inverted diverticulum, whereby the substance gradually reduces a size of and eventually destroys the inverted diverticulum.

10. The method of claim 1, further including the steps of applying an adhesive within the diverticulum and dispensing a filler material into the diverticulum.

11. The method of claim 10, wherein the filler material includes one or more polymers derived from vinyl, acrylate, methacrylate, urethane, ester and oxide monomers, naturally occurring polysaccharides such as chitin, chitosan, dextran and pullulan, gum agar, gum arabic, gum karaya, locust bean gum, gum tragacanth, carrageenans, gum ghatti, guar gum, xanthan gum and scleroglucan; starches, hydrophilic colloids, phosphatides, alginates, gelatin; collagen, cellulosics, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid, homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

12. A method of treating a diverticulum in a body cavity or passageway, the method comprising the steps of:
inserting an endoscope into the cavity;
pulling walls forming the diverticulum toward one another utilizing the endoscope; and
applying adhesive to the walls forming the diverticulum to secure the walls to one another.

13. The method of claim 12, wherein the adhesive includes an antibiotic mixed therein.

14. The method of claim 12, wherein the adhesive is selected from the group consisting of hyaluronic acid, fibrin glue, fibrin clot, blood clot, collagen gel, alginate gel, gelatin-resorcin-formalin adhesive, mussel-based adhesive, dihydroxyphenylalanine (DOPA) based adhesive, chitosan, transglutaminase, poly(amino acid)-based adhesive, cellulose-based adhesive, polysaccharide-based adhesive, synthetic acrylate-based adhesives, platelet rich plasma (PRP), platelet poor plasma (PPP), clot of PRP, clot of PPP, Matrigel, Monostearoyl Glycerol co-Succinate (MGSA), Monostearoyl Glycerol co-Succinate/polyethylene glycol (MGSA/PEG) copolymers, laminin, elastin, proteoglycans, divinyl sulfone (DVS), polyethylene glycol divinyl sulfone (VS-PEG-VS), hydroxyethyl methacrylate divinyl sulfone (HEMA-DIS-HEMA), formaldehyde, glutaraldehyde, aldehydes, isocyanates, alkyl and aryl halides, imidoesters, N-substituted maleimides, acylating compounds, carbodiimide, hydroxychloride, N-hydroxysuccinimide, and mixtures thereof.

15. The method of claim 14, wherein during the step of pulling walls forming the diverticulum toward one another, a vacuum is utilized to pull the walls toward one another.

16. A method of treating diverticula in a body cavity or passageway, the method comprising the step of:
inserting a cylindrical structure into the body cavity such that solid portions of the cylindrical structure align with openings of two or more diverticula;
wherein the solid portions block the openings of the diverticula to prevent material from entering the diverticula.

17. The method of claim 16, further including the step of cleaning the diverticula, wherein the cleaning step includes applying an antibiotic to one or more of the diverticula.

18. The method of claim 16, further including the step of filling one or more of the diverticula with a filler material prior to inserting and aligning the stent with openings of the diverticula.

19. The method of claim 18, wherein the filler material includes one or more polymers derived from vinyl, acrylate, methacrylate, urethane, ester and oxide monomers, naturally occurring polysaccharides such as chitin, chitosan, dextran and pullulan, gum agar, gum arabic, gum karaya, locust bean gum, gum tragacanth, carrageenans, gum ghatti, guar gum, xanthan gum and scleroglucan; starches, hydrophilic colloids, phosphatides, alginates, gelatin; collagen, cellulosics, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid, homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

20. The method of claim 18, wherein the filler material is mixed with an antibiotic.
